(19) United States
(12) Patent Application Publication
(10) Pub. No.: US 2007/0102002 A1
(43) Pub. Date: May 10, 2007

(54) Arrangement for reducing or destroying germs in body cavities of living organisms

(76) Inventor: Christine Schmid, Mistelbach (AT)

Correspondence Address: HENRY M FEIEREISEN, LLC, 350 FIFTH AVENUE, SUITE 4714, NEW YORK, NY, 10118, US

(21) Appl. No.: 10/596,550

(22) PCT Filed: Dec. 15, 2004
(86) PCT No.: PCT/AT04/00440

§ 371 Date: Jun. 16, 2006

(30) Foreign Application Priority Data
Dec. 17, 2003 (AT) 2027/2003

(51) Int. Cl. A61F 6/06

(52) U.S. Cl. 128/830

(57) ABSTRACT

The invention relates to an arrangement for reducing or destroying germs in body cavities, e.g., vagina (1), wherein the arrangement is formed by a shaped body (3) which is introduced into the body cavity and contacts the surrounding tissue and which is made of a medically acceptable metal, e.g. fine silver.

The invention relates to an arrangement for reducing or destroying germs in body cavities of living organisms.

Microorganisms introduced in such body cavities, e.g. the female vagina, can easily reproduce as a consequence of biological conditions like temperature and moisture, causing diseases when the quantity of germs is accordingly. In particular when infections of the female vagina are involved, urinary tract infections and possible premature births in pregnant women are the result.

Reduction of germs in these body cavities is typically treated through oral or local administration of antibiotics or fungicides.

The following invention is now based on the object to find an arrangement of the afore-stated type without straining the organism for preventing as far as possible the occurrence of infections or, if present, to realize a germ reduction or to at least prevent the reproduction of pathogenic germs.

The object is attained in accordance with the invention by implementing the arrangement in the form of a shaped body which is introduced into the body cavities and contacts the surrounding tissue and which is made of a medically acceptable metal, especially fine silver. Silver has a bacteriostatic or bactericide effect as a consequence of its oligodynamic action, wherein sterile silver promotes granulation within the body cavities, when a wound is treated.

When used in the vagina, the shaped body rests advantageously with its outer perimeter upon the inner wall of the vagina for the bactericide effect to take hold. The shaped body may hereby be configured as hollow body, in particular as hollow hemisphere or spiral, to thereby simplify insertion on one hand and to enable a slight weight of the arrangement according to the invention on the other hand. Finally, a thread or a flexible wire, in particular silver wire, may be attached to the shaped body for recapturing the shaped body from the body cavity so that handling, in particular cleansing of the arrangement according to the invention is facilitated.

The drawing shows in

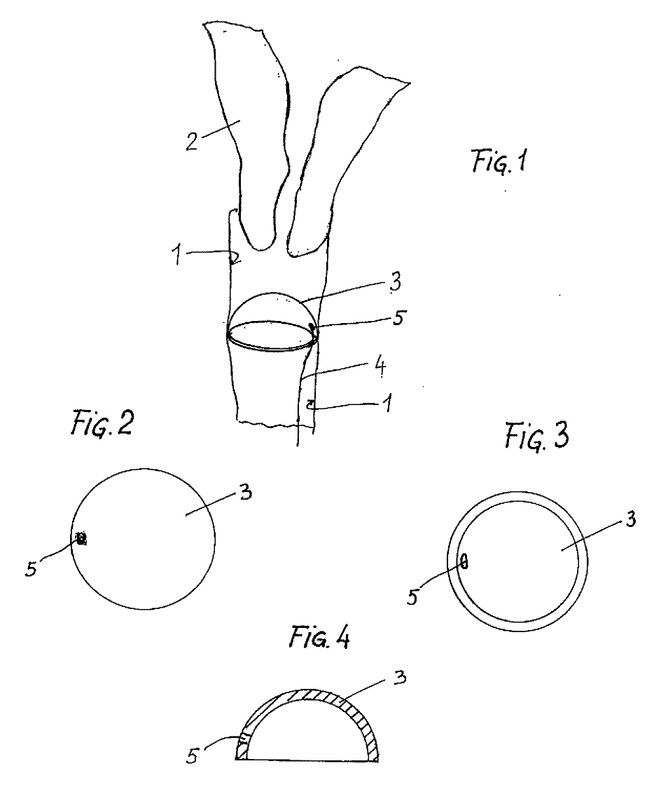

FIG. 1 a highly schematical depiction of an exemplary embodiment of the subject matter of the invention in use.

FIG. 2 shows a plan view of the shaped body according to FIG. 1,

FIG. 3 shows a bottom view, and

FIG. 4 shows a vertical section.

1 designated highly stylized an inner vaginal wall, and 2 the uterus, with the arrangement according to the invention, namely a silver hemisphere 3 for example, being placed in opposition to the uterus. Attached to the silver hemisphere via an opening 5 is a recapture thread or wire 4.

Although the shape of the silver body is secondary, the use of the hemisphere or the spiral has proven advantageous for weight reasons and also with respect to handling during insertion and withdrawal. Of course, any other shape allowing a respective contact with the surrounding tissue can be used.

During an observation period over three months, the arrangement according to the invention, configured as hollow hemisphere made of fine silver, has been placed in the vagina daily for 12 hours (mainly at night) and tested by female volunteers. During this time interval, the test persons experienced no subjective discomfort, like discharge, itching, burning or the like.

It has been found objectively that no antibiotics or antimycotics had to be locally administered.

1-4. (canceled)

5. A structure for preventing infection or reproduction of pathogenic germs or reducing the number of ubiquitously present germs in a body cavity of a living organism, comprising a shaped body made of a medically acceptable material having bacteriostatic or bactericide properties for placement in the body cavity and against a wall of the body cavity.

6. The structure of claim 5, wherein the body cavity is a female vagina.

7. The structure of claim 5, wherein the shaped body is a hollow body.

8. The structure of claim 5, wherein the shaped body is a hollow hemisphere.

9. The structure of claim 5, wherein the shaped body is made of metal.

10. The structure of claim 5, wherein the shaped body is made of fine silver.

11. The structure of claim 5, further comprising a recapture member attached to the shaped body (3) for retrieving the shaped body from the body cavity.

12. The structure of claim 5, wherein the recapture member is a thread.

13. The structure of claim 5, wherein the recapture member is a flexible wire.

14. The structure of claim 5, wherein the recapture member is a silver wire.
